(19) United States
(12) Patent Application Publication
(10) Pub. No.: US 2003/0134270 A1
(43) Pub. Date: Jul. 17, 2003

(54) Rapid method of selecting cells for gene disruption by homologous recombination

(76) Inventors: Robert M. Burgess, JR., Temecula, CA (US); Patrick A. Schneider, Temecula, CA (US)

Correspondence Address: GRAY CARY WARE & FRIEDENRICH LLP, 4365 EXECUTIVE DRIVE, SUITE 1100, SAN DIEGO, CA, 92121-2133, US

(21) Appl. No.: 10/345,130

(22) Filed: Jan. 14, 2003

Related U.S. Application Data
(60) Provisional application No. 60/348,549, filed on Jan. 14, 2002.

(51) Int. Cl. C12Q 1/00; C12Q 1/68; C12N 15/85

(52) U.S. Cl. 435/4; 435/6; 435/455

(57) ABSTRACT

Procedures and vectors are provided for the specific alteration of particular genetic loci in eukaryotic cells. One procedure comprises the utilization of ATG-minus fluorescent protein gene targeting (AMFP) DNA vectors for the purpose of creating and identifying cells which have vector sequences integrated into the host cell genome via site-specific homologous recombination. The procedure also comprises the utilization of sequences encoding in vivo detectable markers for the identification of cells which have exogenous vector sequences integrated into the genome of the host cell, either via site-specific homologous recombination or nonhomologous recombination or insertion. The invention also includes vectors for creating modifications in eukaryotic cells. In addition, the invention includes cells and organisms generated from cells with specific genetic alterations through the implementation and use of provided procedures and vectors.

### RELATED APPLICATION DATA

This application claims the benefit of priority to under 35 U.S.C. § 119(e)(1) of U.S. Ser. No. 60/348,549, filed Jan. 14, 2002, the entire context of which is incorporated herein by reference.

### BACKGROUND OF THE INVENTION

1. Field of the Invention

The present invention relates generally to the manipulation of cells for the purposes of modifying genetic loci and more specifically, the invention relates to vectors and methods for generating genetic modifications in cells.

2. Background Information

Stable introduction of foreign genetic material into the genomes of both prokaryotic and eukaryotic organisms has been successfully accomplished in a variety of instances for various purposes such as the expression of an exogenous gene or the disruption of an endogenous locus. It is accomplished primarily through either random genomic insertion or site-specific homologous recombination. Random integration involves the insertion of a linearized DNA fragment into the genome of the host cell at locations which are, for the most part, non-site-specific. These insertions tend to exist as multimers or concatemers and most often do not result in the disruption and inactivation of a particular locus. The possibility also exists that endogenous loci may be disrupted by the random insertion event, thus often making analysis of the exogenous gene's effects on the cell or organism derived from the transformed cell difficult. In addition, a significant range of exogenous promoter activity may be observed depending upon the region of integration.

Insertion of DNA into the host genome via site-specific homologous recombination allows for the targeting of particular regions of the host genome for single copy integration of the exogenous DNA. Homologous recombination involves the exchange of significantly similar nucleotide sequences through the function of specific recombinase enzymes. Early experiments designed to manipulate cellular endogenous genomic DNA sequences with exogenous DNA in a site-specific manner focused on yeast as a model system. Recombination was demonstrated between the yeast genome and an exogenous plasmid introduced via transformation at the leu2.sup locus (Hinnen et al. (1978), Proc. Natl. Acad. Sci. U.S.A., 75, 1929). More recently the utilization of mammalian cellular homologous recombination capacities has allowed for the generation of specific mutated DNA sequences within the cellular endogenous genomic DNA. Both gain-of-function and loss-of-function alleles have been generated in stem cells and animals generated from said cells (see below). In addition, the application of positive-negative selection vectors and methods has accelerated the generation and study of cells and animals containing mutated DNA sequences (Capecchi et al. (1997) U.S. Pat. No. 5,631,153). To date, primarily two types of vectors have been designed which allow for targeting of a specific region of the genome for replacement of endogenous with exogenous DNA sequences. These vectors have proven to be sufficient for the generation of a variety of targeted alleles in a number of different cell types.

Insertion vectors contain two regions of homology flanking an internal nucleotide sequence encoding a selectable marker. The vector is linearized within one of the regions of homology. A single crossover event and homologous recombination results in a partial duplication of genomic sequences. Intrachromosomal recombination often results in exclusion of the endogenous duplicated sequences. A disadvantage to this type of targeting vector is the lack of a negative selectable marker which would allow for significant enrichment for correctly targeted events through elimination of cells which contain backbone or vector sequences. In addition, linearization within a region of homology reduces the amount of DNA sequence available for homologous recombination thus reducing the opportunity for strand exchange (Thomas et al. (1986), Cell, 44, 49). Finally, intrachromosomal recombination must occur within a defined region or regeneration of the wild-type organization of the locus may occur.

Replacement vectors contain two regions of homology usually flanking a positive selectable marker, such as the gene encoding neomycin phosphotransferase. A negative selectable maker is often located external but adjacent to one of the regions of homology to provide for enrichment of corrected targeted cell in the total population through elimination of cells containing the negative selectable cassette. Introduction of a replacement vector into cells followed by simultaneous or stepwise positive and negative selection results in the isolation of cells which have perhaps an eight to twelve-fold enriched probability of undergoing site-specific homologous recombination due to application of the negative selectable marker. In perhaps the first successful gene targeting experiments in mammalian cells, Capecchi et al. have demonstrated targeting of the mouse HPRT and int-2 loci via the use of replacement vectors (Capecchi et al., (1997), U.S. Pat. No. 5,631,153). A plethora of loci have since been successfully targeted, some by insertion vectors and the majority by replacement vectors. Many of these have included a negative selectable marker positioned external to either or both of the regions of homology, which often results in an increase in the efficiency of targeted allele identification. Yet a number of disadvantages exist with respect to the method and utility of replacement vectors and positive-negative selection. Utilization of a number of negative selectable cassettes such as HSV thymidine kinase requires the addition of an antibiotic or selective agent, gancyclovir for example, which may cause undo stress to the cells and unwanted or premature differentiation. In addition, selection of cells for enrichment with a negative selectable marker takes considerable time to allow for the cells to recover which have resistance to the drug due to absence of the selectable marker. As well, the enrichment factors typically obtained by this methodology are at most between eight to twelve-fold. As well, the creation of positive-negative selection vectors is often strategically difficult and time-consuming.

Alternative positive selection strategies have been designed which do not include the utilization of positive-negative selection. These include the application of strategies for the conditional expression of a dominant selectable marker by virtue of in-frame gene fusion with the target gene. Utilizing the resistance marker neomycin phosphotransferase Sedivy et al. demonstrated successful gene targeting of the polyoma middle T antigen (pmt) locus (Sedivy et al. (1989), Proc. Natl. Acad. Sci., 86, 227). The implementation, however, of a drug selectable resistance marker for the detection of site-directed homologous recombination events is invasive and toxic to the cells undergoing selection. The technology described in the present invention circumvents these issues.

A number of animals have also been created from embryonic stem cells which have particular loci mutated through site-specific homologous recombination. These include mice which are derived from chimeras produced by injection of blastocysts with embryonic stem cells targeted through homologous recombination at particular loci. Some examples include the p53 and paraxis loci (Donehower et al. (1992), Nature, 356, 215; Burgess et al. (1996), Nature, 384, 570). Pigs have also been derived from embryonic stem cells modified by homologous recombination include pigs (Butler et al. (2002), Nature, 415, 103).

### SUMMARY OF THE INVENTION

Methods are provided for the modification of genomic DNA sequences through homologous recombination of vector DNA with target DNA in eukaryotic cells. The methods entail first the transformation of a cell capable of undergoing homologous recombination with a vector, referred to herein as a ATG-minus fluorescent protein gene targeting vector (AMFP) containing sequences substantially similar to sequences present within the genome of the cell (FIGS. 1, 2 and 3). The majority of the vector integration into the genome of the host cell will occur in an essentially random manner, with no preference for particular regions of the genome. It is reasonably suggested, however, that a certain percentage of the AMFP gene targeting vector will integrate into the genome of the host cell via site-specific homologous recombination. Subsequent selection of the cells will allow for the isolation and identification of cells which have successfully undergone site-specific homologous recombination (FIG. 4). The selection is based upon the organization and composition of the AMFP replacement vector.

The vector is composed of a first DNA sequence which is significantly homologous to a sequence present within the host cell genome. In addition, the vector includes a third DNA sequence which is significantly homologous to other sequences within the host cell genome downstream or upstream of the first sequence. The vector contains between these two regions a second DNA sequence which is not significantly homologous to sequences present in the host genome and confers the ability to identify cells which have vector sequences integrated into said genome. It is the utilization of the second DNA sequence that allows for the identification of cells which have undergone homologous recombination of the vector with endogenous sequences. In addition, the invention includes cells and organisms generated from cells with specific genetic alterations through the implementation and use of provided procedures and vectors.

In a first embodiment, the invention provides a method for identifying a transformed cell which has undergone site-specific homologous recombination utilizing an AMFP gene targeting vector. The method includes:

a) transforming cells with an AMFP gene targeting vector designed to undergo site-specific homologous recombination wherein the vector includes:

a first DNA sequence which is substantially homologous to an endogenous genomic sequence present within the host genome;

a second DNA sequence which lacks regulatory elements sufficient to drive its expression and encodes a fluorescent protein selectable marker lacking nucleotide sequences coding for an initiating methionine in said cells and is non-homologous to cellular endogenous genomic sequences and therefore incapable of undergoing site-specific homologous recombination;

a third DNA sequence which is substantially homologous to an endogenous genomic sequence present within the host genome and is different from the first DNA sequence;

wherein the vector is capable of undergoing site-specific homologous recombination in cells through strand exchange between the first DNA sequence with endogenous genomic target sequences and the third DNA sequence with endogenous genomic target DNA sequences;

wherein the organization of the DNA sequences in the AMFP vector is: the first DNA sequence which is substantially homologous to target DNA sequences, the second DNA second which lacks regulatory elements sufficient to drive its expression and encodes a fluorescent protein selectable marker lacking nucleotide sequences coding for an initiating methionine and the third DNA sequence which is substantially homologous to target DNA sequences;

b) propagating cells to select for or enrich for those which have been successfully transformed with said AMFP vector and have successfully undergone site-directed homologous recombination by selecting for the presence of the functional fluorescent protein selectable marker gene product of said second DNA sequence, and

c) separating cells which have said second DNA sequence encoding a fluorescent protein selectable marker expressed and translated to produce a functional protein product from cells which do not contain said second DNA sequence or do not effectively express a functional fluorescent protein product. The method may further include

d) characterizing the genomic DNA of said cells carrying the second DNA sequence encoding a functional fluorescent protein selectable marker for the site-specific homologous recombination events which allow for modification of the cellular target DNA.

An object of the present invention is to provide site-specific homologous recombination methods for the targeting of specific regions of eukaryotic genomes for the purposes of modifying endogenous nucleotide sequences.

It is another object of the present invention to provide novel methods for the selection and detection of cells which have undergone site-specific homologous recombination.

It is a further object of the present invention to provide novel vectors for the application of the described methods.

It is still a further object of the present invention to provide cells which have been modified by site-specific homologous recombination methods described.

It is yet another embodiment of the present invention to provide transgenic animals and plants which have been modified by the site-specific homologous recombination and detection methods described.

### BRIEF DESCRIPTION OF FIGURES

FIG. 1 is a diagrammatic illustration of double crossover homologous recombination replacement AMFP targeting of a theoretical genomic locus utilizing target exonic sequences 5′ of the internal fluorescent protein selectable marker with GFP encompassed in the same exon.

FIG. 2 is a diagrammatic illustration of double crossover homologous recombination replacement AMFP targeting of a theoretical genomic locus utilizing target intronic sequences 5′ of the internal positive selectable marker with GFP encompassed in a downstream exon.

FIG. 3 is a diagrammatic illustration of double crossover homologous recombination replacement AMFP targeting of a theoretical genomic locus utilizing target intronic and exonic sequences 5′ and 3′ of the internal positive selectable marker with GFP encompassed internally within an intron in combination with consensus splice acceptor and splice donor sites.

FIG. 4 is a flowchart representation of the process implemented for achieving site-directed homologous recombination via AMFP gene targeting.

FIG. 5 is a diagrammatic illustration of double crossover homologous recombination replacement AMFP targeting of the ptch 2 genomic locus.

FIG. 6 is a diagrammatic illustration of single crossover homologous recombination replacement AMFP targeting of the paraxis genomic locus.

### DETAILED DESCRIPTION OF THE INVENTION

The methods and vectors described in the present invention are utilized for the purpose of introducing modifications into cellular endogenous genomic target DNA sequences via site-specific homologous recombination.

The term “cellular endogenous genomic DNA sequence” is defined herein as nucleotide sequences present within the cellular genome which are capable of undergoing site-specific homologous recombination and may be utilized as a target for modification by the AMFP gene targeting vectors described herein. Sequences included within this definition may represent any coding or noncoding regions of specific genes present within the cellular genome. Genes encoding such protein products as structural proteins, secreted proteins, hormones, receptors, enzymes, transcription factors are included in this definition. These sequences may also represent regulatory element identity such as promoters, enhancers or repressor elements. The organization of the cellular endogenous genomic target DNA sequence is generally similar to specific sequences present within the AMFP gene targeting vector. That is, it contains sequences which are substantially homologous to sequences present within the AMFP gene targeting vector that allow for site-specific homologous recombination to occur.

The term “site-directed homologous recombination” refers to strand exchange crossover events between DNA sequences substantially similar in nucleotide composition. These crossover events may take place between sequences contained in the AMFP gene targeting vector and cellular endogenous genomic DNA sequences. In addition, it is possible that more than one site-specific homologous recombination event may occur between DNA sequences present in the AMFP gene targeting vector and cellular endogenous genomic sequences which would result in a replacement event in which DNA sequences contained within the AMFP gene targeting vector have replaced specific sequences present within the cellular endogenous genomic sequences. As well, a single site-specific homologous recombination event may occur between DNA sequences present in the AMFP gene targeting vector and cellular endogenous genomic sequences which would result in an insertion event in which the majority or the entire AMFP gene targeting vector is inserted at a specific location within the cellular endogenous genomic sequences.

The term “first DNA sequence” refers to DNA sequences present within the AMFP gene targeting vector which are substantially homologous to cellular endogenous genomic sequences. It is these sequences which are predicted to undergo site-specific homologous recombination upon their introduction into cells capable of undergoing said recombination which contain similar sequences.

The term “second DNA sequence” refers to sequences encoding a fluorescent protein selectable marker which lacks nucleotides sequences coding for an initiating methionine and which does not contain a promoter or regulatory elements driving the expression of said fluorescent protein selectable marker. The selectable marker is positioned between the first and third DNA sequences which are substantially homologous to cellular endogenous genomic DNA sequences. The selectable marker is nonhomologous to cellular endogenous genomic DNA sequences and therefore incapable of site-specific homologous recombination with these sequences.

The term “third DNA sequence” refers to DNA sequences present within the AMFP gene targeting vector which are substantially homologous to cellular endogenous genomic sequences yet are different but possibly adjacent or within reasonable proximity to those of the first DNA sequence. It is these sequences which are predicted to undergo site-specific homologous recombination upon their introduction into cells capable of undergoing said recombination which contain similar sequences.

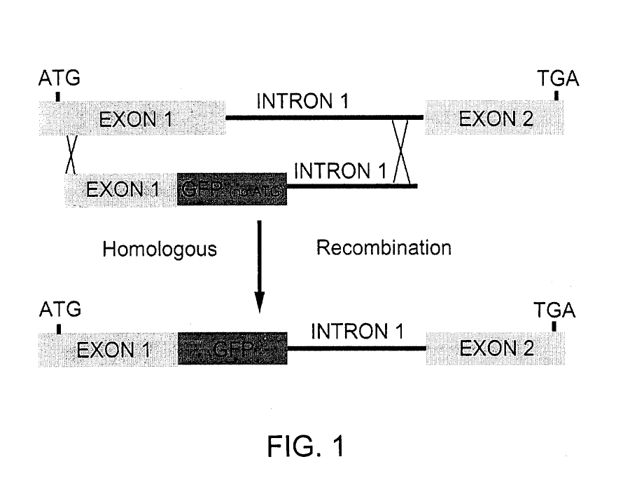

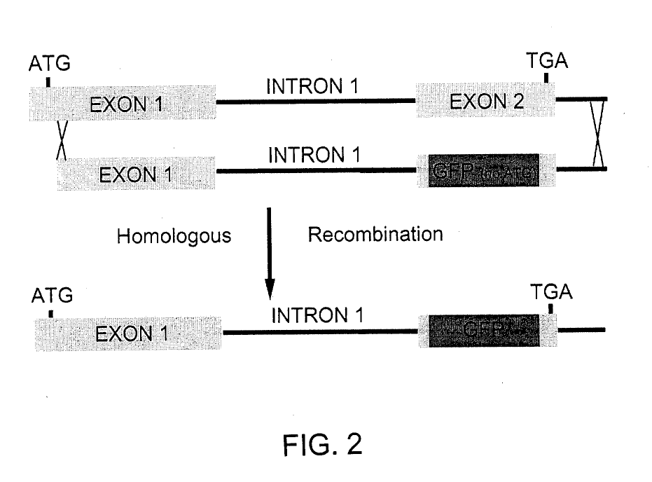

In a replacement AMFP gene targeting vector, the first, second and third DNA sequences are organized such that the second DNA sequence, which encodes a fluorescent protein selectable marker lacking nucleotide sequences coding for an initiating methionine, is positioned between the first and third DNA sequences. FIGS. 1, 2 and 3 illustrate the organization of three different types of AMFP gene targeting vectors utilized for site-specific homologous recombination.

Upstream generally refers to 5′ and downstream generally refers to 3′ of the first and third DNA sequences in a vector which has both the first and third DNA sequences in an orientation similar to that of cellular endogenous genomic sequences. It is to be clarified that 5′ and 3′ refer to the first and third DNA sequences respectively. This organization represents a replacement vector. In addition, it is possible that portions of the first and third sequences in the AMFP gene targeting vector are inverted with respect to one another in comparison to similar sequences in the cellular target DNA. This type of organization represents an insertion vector. Insertion vectors generally incorporate the majority of the vector sequence into the cellular genome upon site-specific homologous recombination.

In an insertion AMFP gene targeting vector, the first, second and third sequences are organized such that the third sequence has an inverted 5′ to 3′ orientation with respect to the first sequence upon linearization of the vector. Said inverted orientation allows for the insertion of the vector at a site-specific location upon site-specific homologous recombination between the AMFP gene targeting vector and cellular endogenous genomic DNA sequences. In the majority of the cases the entire vector will be inserted and portions of the substantially homologous DNA sequences duplicated.

The length of the AMFP gene targeting vector will vary depending upon the choice of fluorescent protein selectable marker, the length of the first and third DNA sequences required for appropriate homologous recombination, the size of the base vector and the choices for selection of the plasmid vector in bacteria such as ampicillin resistance and the size of the origin of replication for the plasmid backbone. It is reasonably estimated, however, based upon the sizes of known plasmids and positive selectable markers, that the entire vector will be at least several kilobasepairs in length.

The term “functional” is defined herein with respect to fluorescent protein selectable markers as conferring the ability of markers to allow for the detection and isolation of cells containing DNA encoding the fluorescent protein selectable marker and to allow for the differentiation of these cells from cells which contain either no fluorescent protein selectable marker or a fluorescent protein selectable marker which is unique in comparison to the first fluorescent protein selectable marker. In the presently described invention fluorescent protein selectable markers are considered non-functional if the corresponding coding nucleotide sequences lack sequences coding for an initiating methionine. It is only upon recombination with endogenous genomic target sequences that the acquisition of nucleotide sequences coding for an initiating methionine and acquisition of regulatory elements sufficient to drive the expression and translation of the fluorescent protein selectable marker occur thus making the fluorescent protein selectable marker functional.

TABLE I
Fluorescent Protein Selectable Markers Utilized in AMFP Vectors

| Fluorescent Protein Marker | Method for Detection |
|---|---|
| GFP | Fluorescence |
| CFP | Fluorescence |
| YFP | Fluorescence |
| RFP | Fluorescence |
| dsRED | Fluorescence |
| HcRED | Fluorescence |

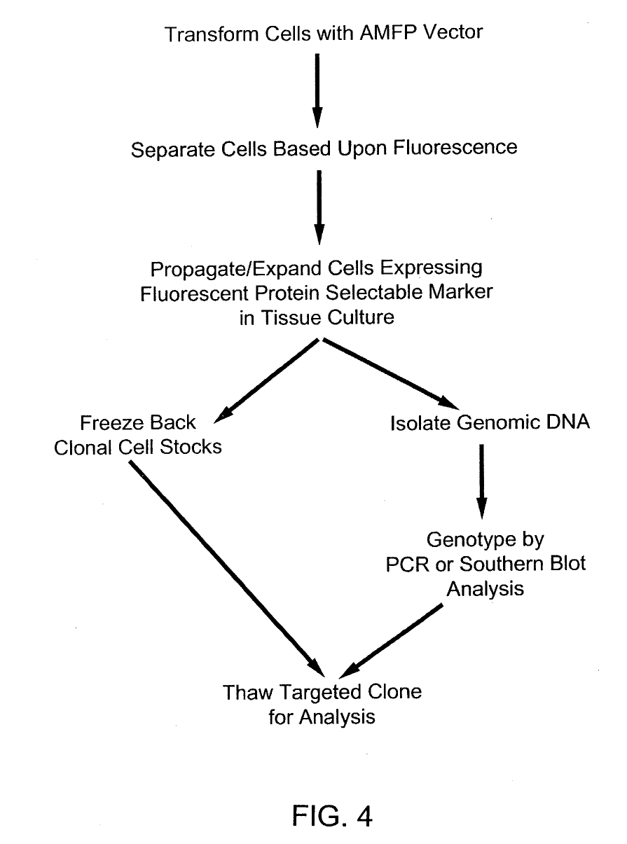

The AMFP gene targeting vector includes two regions of homology, DNA sequences one and three, which are substantially homologous to regions of the host genome. Typically, the vector has lengths of homology for the first and third DNA sequences which are between about 50 base pairs and 50,000 base pairs. It also includes DNA sequence two, which encodes a fluorescent protein selectable marker that allows for the identification of the presence or absence of the AMFP vector integrant and portions thereof within the host genome. The second DNA sequence encodes a fluorescent protein selectable marker lacking sequences coding for an initiating methionine, such as, but not limited to, cyan fluorescent protein (CFP) for example, and is positioned between the two regions of homology, thus it will be included in the host genome integrant should site-specific homologous recombination occur. The selection process involves sorting of cells either under a microscope or through a FACS cell sorting apparatus which will allow for the simultaneous and separate isolation of cells which contain the second DNA sequences encoding a functional fluorescent protein selectable marker from cells which do not contain said marker in a functional capacity. Cells may subsequently be propagated in tissue culture and genotyped for correct site-specific homologous recombination gene targeting events (FIG. 4). Given the noninvasive nature of the described AMFP gene targeting methodologies, the utilization of fluorescent protein selectable markers for the isolation of cells which have undergone site-specific homologous recombination allows for a substantial improvement over existing methodologies for gene targeting.

The AMFP gene targeting vectors utilized in the presently described invention are organized such that the second DNA sequence which encodes a fluorescent protein selectable marker lacking sequences coding for an initiating methionine is operatively positioned between the two regions of homology. It is possible that the second DNA sequence may be positioned in such a fashion as to disrupt or replace exonic or coding sequences of the endogenous region of the genome at which site-specific homologous recombination may occur thus rendering the endogenous locus inactive and thus nonfunctional (FIG. 1).

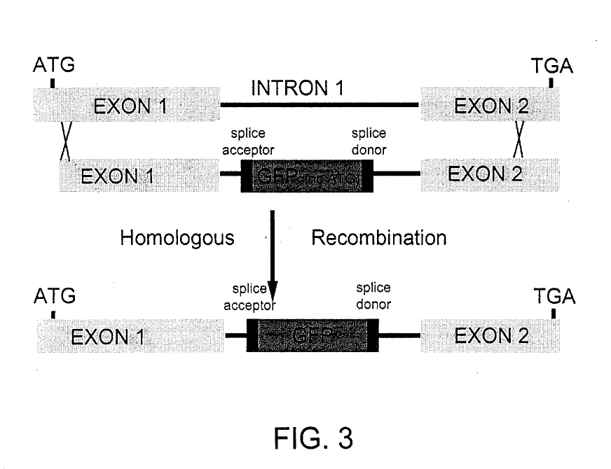

In one aspect, the second DNA sequence may be positioned such that it replaces or inserts into regions of the genome which do not confer exonic or coding sequences such as introns, untranslated regions of exons or regulatory element regions such as promoters. In this scenario it may be possible to select for cells which have undergone site-specific homologous recombination at the locus without inactivating that particular locus (FIG. 3).

The presently described invention also includes cells which have undergone site-specific homologous recombination in accordance with the AMFP gene targeting vectors and methods for identification described herein.

In addition, the presently described invention includes transgenic non-human animals which have been derived from cells which have undergone site-specific homologous recombination utilizing AMFP gene targeting vectors and methods described herein.

Also included are transgenic plants which have been derived from cells which have undergone site-specific homologous recombination utilizing AMFP gene targeting vectors and methods described herein. Plants have previously been demonstrated to undergo site-specific homologous recombination as well as gene targeting via positive-negative selection and are therefore amenable to the AMFP gene targeting vectors and methods described herein (Siebert et al. (2002), Plant Cell, 14, 1121; Hanin et al. (2001), Plant J., 28, 671; Xiaohui et al. (2001), Gene, 272, 249).

A number of nucleotide sequences coding for preferred fluorescent protein selectable markers exist for the second DNA sequence (Table I). These sequences allow for selection of cells carrying the fluorescent protein selectable marker in order to distinguish said cells from those which do not carry the fluorescent protein selectable marker or cells which do not effectively express a functional version of the fluorescent selectable marker protein product. Perhaps the most widely utilized fluorescent protein selectable marker utilized as the second DNA sequence encodes the green fluorescent protein gene product (Prascher et al. (1992), Gene, 15, 229). Other fluorescent protein selectable markers appropriate for the second DNA sequence include, but are not limited to, those which code for red fluorescent protein (RFP), cyan fluorescent protein (CFP) and yellow fluorescent protein (YFP). Several of these fluorescent protein selectable markers may also be applied to the use of AMFP gene targeting vectors for site-specific homologous recombination in plants.

A “mutating DNA sequence” is herein referred to as any sequence which changes the nucleotide composition of cellular endogenous genomic DNA sequences. Said change may result in an inactivation of the functional capacity of the cellular DNA sequence. Said change may also enhance the functional capacity of the cellular DNA sequence or it may have no effect on the functional capacity of the cellular DNA sequence.

A “mutated DNA sequence” is herein referred to as any cellular endogenous genomic DNA sequence which has undergone alteration through the utilization of AMFP gene targeting vectors. It is generally anticipated that mutated DNA sequences will be generated upon site-specific homologous recombination between the AMFP gene targeting vector and cellular endogenous genomic DNA sequences.

“Mutated target cells” are cells capable of undergoing site-specific homologous recombination which have a mutated DNA sequence established within the cellular genome through the application of mutating DNA sequences present in the AMFP gene targeting vectors described herein.

The term “substantially nonhomologous DNA” refers to DNA sequences which do not contain nucleotide sequences similar enough to target DNA sequences to allow for the process of site-specific homologous recombination to occur. Dissimilar sequences of this capacity fail to undergo site-specific homologous recombination with target DNA sequences due to the mismatch of base pair composition between the two sequences.

There are a number of applicable advantages to establishing mutated DNA sequences within a cellular genome. X-linked genes, for example, may be analyzed for functional relevance in tissue culture if the particular cell type targeted by the AMFP gene targeting vector is of male origin. In addition, manipulation of embryonic stem cells via AMFP gene targeting vectors may allow for the creation of animal models for the study of human disorders. The p53 locus, for example, has been successfully inactivated via positive-negative selection technology in mouse embryonic stem cells and those cells utilized for the creation of mice deficient in the protein product encoded by this locus (Donehower et al. (1992), Nature, 356, 215). These mice are developmentally normal but susceptible to spontaneous tumors. AMFP gene targeting vectors and technology allow for the generation of similar genetic modifications in embryonic stem cells and animals created from said cells. Other uses of AMFP gene targeting vectors and technology include the generation of gain-of-function alleles which may allow for the study of a variety of cellular and physiologic phenomena. Many proto-oncogenes have been analyzed as gain-of-function alleles including c-myc, cyclin D1 and ErbB-2 (for review see Hutchinson et al. (2000), Oncogene, 19, 130). Use of the AMFP gene targeting vectors and methods described herein efficiently allow for both loss- and gain-of-function studies in embryonic stem cells as well as transgenic animals derived from these cells.

AMFP gene targeting vectors and methods are utilized for the purposes of creating and identifying cells which have undergone site-specific homologous recombination between the vector and cellular endogenous genomic target sequences. The vectors substantially enrich for the identification of cells which have undergone said process. To “substantially enrich” refers to the ability to significantly increase the likelihood of identifying cells for which site-specific homologous recombination between the vector and cell DNA sequences. The significant increase in likelihood is at least two-fold of homologous recombination events when compared to nonspecific insertion or integration events, preferably at least 10-fold, more preferably at least 100-fold and even more preferably at least 10,000-fold. Substantially enriched cell populations derived from the use of AMFP gene targeting vectors include around 1%, more preferably 10%, and even more preferably 99% of cells isolated have undergone site-specific homologous recombination between AMFP gene targeting vector sequences and cellular endogenous genomic target sequences.

The AMFP gene targeting vectors and methodology described herein may be utilized for the purposes of correcting specific genetic defects in humans. It is possible, for example, to generate a mutated DNA sequence in human stem cells through site-specific homologous recombination between an AMFP gene targeting vector and cellular endogenous genomic DNA sequences and subsequently transplant those cells into patients for the correction of a specific genetic disorder or supplementation of a particular gene product. Another potential use for gene inactivation is disruption of proteinaceous receptors on cell surfaces. For example cell lines or organisms wherein the expression of a putative viral receptor has been disrupted using an appropriate AMFP gene targeting vector can be assayed with virus to confirm that the receptor is, in fact, involved in viral infection. Further, appropriate AMFP gene targeting vectors may be used to produce transgenic animal models for specific genetic defects. For example, many gene defects have been characterized by the failure of specific genes to express functional gene product, e.g. .alpha. and .beta. thalassema, hemophilia, Gaucher's disease and defects affecting the production of .alpha.-1-antitrypsin, ADA, PNP, phenylketonurea, familial hypercholesterolemia and retinoblastemia. Transgenic animals containing disruption of one or both alleles associated with such disease states or modification to encode the specific gene defect can be used as models for therapy. For those animals which are viable at birth, experimental therapy can be applied. When, however, the gene defect affects survival, an appropriate generation (e.g. F0, F1) of transgenic animal may be used to study in vivo techniques for gene therapy.

AMFP gene targeting vectors are designed for the specific purposes of mutating DNA sequences in the endogenous genomic DNA of cells capable of undergoing site-specific homologous recombination. The components of the AMFP gene targeting vector include at least one region of DNA which is substantially homologous to cellular endogenous genomic DNA sequences and one DNA sequence encoding a fluorescent protein selectable marker lacking regulatory elements or nucleotide sequences coding for an initiating methionine but capable of conferring the ability to identify cells containing the fluorescent protein selectable marker from cells which do not contain sequences encoding the fluorescent protein selectable marker upon site-specific homologous recombination with endogenous genomic target sequences (FIG. 1).

In addition, it is preferable that the AMFP gene targeting vector be linearized prior to its introduction into cells for the purposes of mutating cellular endogenous genomic DNA sequences as linear vectors exhibit significantly higher targeting frequencies than those circular (Thomas et al. (1986), Cell, 44, 49). It is, however, possible to successfully utilize AMFP gene targeting vectors for these purposes without linearization.

For the purposes of targeting different alleles, it may be necessary to utilize different fluorescent protein selectable markers. By manipulating the identity of the fluorescent protein selectable markers different alleles may be targeted successfully either simultaneously or sequentially.

The length of the AMFP gene targeting vector required for successful site-specific homologous recombination is a critical parameter that is often dependent upon the particular gene targeted for creating a mutated DNA sequence. Vector length is dependent upon several factors. The choice of the DNA sequences encoding the fluorescent protein selectable markers will affect the overall vector length due to the variation of sequence composition for different markers. In addition, in a replacement vector the lengths of DNA sequences one and three, the two sequences which are substantially homologous to cellular endogenous genomic target DNA sequences, are crucial parameters that must be correctly addressed for successful gene targeting. In general, one region of homology may be as small as 25 bp (Ayares et al. (1986), Genetics, 83, 5199), although it is recommended that significantly larger regions of homology be utilized. Up to a certain length, an increase in the amount of homology provided in the AMFP gene targeting vector increases targeting efficiency (Zhang et al. (1994), Mol. Cell Biol., 14, 2404). In most cases the entire vector length will be a minimum of 1 kb and usually will not exceed a maximum of 500 kb, although vector length is also dependent upon the technology utilized to construct the vector. It is possible, for example, to construct a AMFP gene targeting vector with a cosmid, BAC, or YAC as the provider of the two regions of substantial homology thus generating a significantly large vector (Ananvoranich et al. (1997), Biotechniques, 23, 812; Cocchia et al., (2000), Nucleic Acids Res., 28, E81). Vector length also includes plasmid backbone sequences such as those encoding the origin of replication and bacterial drug resistance products such as ampicillin if these are not removed prior to transformation of cells with the vector.

AMFP gene targeting vector DNA sequences which are substantially homologous to cellular endogenous genomic DNA sequences and undergo site-specific homologous recombination for the purpose of creating mutated DNA sequences in cellular targets are preferred to have significantly high homology to cellular counterparts. High homology allows for efficient base pairing during the crossover and strand exchange process of site-specific homologous recombination. Any mismatch base pairing between AMFP gene targeting vector and cellular DNA sequences disfavors the recombination reaction. It is preferable, for example, that DNA sequences one and three in a AMFP gene targeting replacement vector are 100% homologous to cellular endogenous genomic DNA sequences, less preferable that they are 80% homologous and even less preferable that they are 50% homologous. The second which encodes a fluorescent protein selectable marker is generally nonhomologous to cellular endogenous genomic DNA sequences and therefore does not undergo site-specific recombination with these sequences.

In certain cases it may be advantageous to remove DNA sequences encoding fluorescent protein selectable markers which have been incorporated into the genome of cells upon site-specific homologous recombination between AMFP gene targeting vectors and cellular endogenous genomic target DNA sequences. This is due to the potential negative effects expression of the fluorescent protein selectable marker may have on cellular or organismal viability and survival. The removal of sequences encoding fluorescent protein selectable markers is possible by a number of methodologies. The Cre-Lox technology may be successfully applied for the removal of specific sequences introduced into cellular endogenous genomic DNA via AMFP gene targeting vectors and technology (for review on Cre-Lox see Ryding et al. (2001), J Endocrinol., 171, 1). For example, sequences encoding a fluorescent protein selectable marker may be flanked with LoxP recombination sites in the AMFP gene targeting vector prior to cellular transformation. After introduction of these sequences into the genome of the host cell a transient or stable expression of the Cre recombinase will allow for removal of one LoxP site and all sequences positioned between the LoxP sites. Many examples of the application of Cre-lox technology for sequence removal exist. Kaartinen et al. have demonstrated removal of a neomycin phosphotransferase cassette flanked by lox P site through the transient expression of Cre via adenoviral infection of 16-cell-stage morulae (Kaartinen et al. (2001), Genesis, 31, 126). Xu et al. successfully removed a lox P flanked neomycin phosphotransferase cassette through both a cross with mice expressing Cre under the control of the EIIa promoter as well as pronuclear injection of cells containing the cassette with a Cre-expressing plasmid (Xu et al. (2001), Genesis, 30, 1). Thus, if the AMFP gene targeting vector is configured to replace or correct cellular exonic sequences which are defective, such as may be the case for human gene therapy, the fluorescent protein selectable marker may be removed after completion of site-specific homologous recombination between the AMFP gene targeting vector and host DNA.

The AMFP gene targeting vectors and methodology described herein may also be utilized for the purposes of mutating DNA sequences in plants. Indeed, several examples of homologous recombination in plant lineages exist (Siebert, et al. (2002), Plant Cell, 14, 1121 and for review see Schaefer, D. G. (2002), Annu. Rev Plant Physiol. Plant Mol Biol., 53, 477). In addition, said homologous recombination has been exploited utilizing positive-negative selection technology to target several plant loci including the alcohol dehydrogenase and protoporphyrinogen oxidase (PPO) loci (Xiaohui et al., (2001), Gene, 272, 249; Hanin et al., (2001), Plant J, 28, 671). It is postulated that there are a number of fluorescent protein markers which may be utilized for the purposes of implementing AMFP gene targeting methodology to generate mutated DNA sequences via site-specific homologous recombination (Table I). Mutations in plants created utilizing AMFP gene targeting vectors and methodology may encompass loss-of-function, gain-of-function or modifications in the expression levels of endogenous loci through the introduction of exogenous regulatory elements. Loss-of-function or gain-of-function mutations may be generated through the ablation of specific endogenous DNA sequences or the alteration of sequences which may change the amino acid composition encoded by a particular plant gene. In addition, “knockin” experiments may be performed in plants through the use of AMFP gene targeting vectors and methodology to introduce an exogenous gene or coding region into an endogenous locus.

Introduction of the AMFP gene targeting vector into plant cells may be accomplished by a variety of methods including those previously developed for the insertion of exogenous DNA into protoplasts (Hain et al. (1985), Mol. Gen. Genet., 199, 161; Negrutiu et al. (1987), Plant Mol. Bio., 8, 363; Paszkowski et al. (1984), EMBO J., 3, 2717). Microinjection may also allow for the successful introduction of the AMFP gene targeting vector into plant cells (De la Pena et al. (1987), Nature, 325, 274; Crossway et al. (1986), Mol. Gen. Genet., 202, 179). In addition, it is possible to introduce the AMFP gene targeting vector into plant cells via liposome-mediated transfection (Deshayes et al. (1985), EMBO J., 4, 2731). Upon successful introduction of the AMFP gene targeting vector into plant cells site-specific homologous recombination may allow for the mutation of cellular endogenous genomic DNA sequences according to the construction and organization of the AMFP gene targeting vector.

The cell separation strategies described in the present invention include cell sorting through the utilization of a FACStar Plus cell sorter as well as manual separation techniques, but the invention is not limited to this apparatus or to these separation techniques. Other cell sorting apparatuses may also be implemented for the effective separation of cells which express one selectable marker verses another selectable marker or no selectable marker. These include, but are not limited to, the FACS Vantage SE I, and FACS Vantage SE II or any apparatus capable of sorting cells based upon methods described in the present invention.

The AMFP gene targeting vector is used in the AMFP gene targeting method to select for transformed target cells containing the positive selection marker. Such ATG-minus fluorescent protein gene targeting procedures substantially enrich for those transformed target cells wherein homologous recombination has occurred. As used herein, “substantial enrichment” refers to at least a two-fold enrichment of transformed target cells as compared to the ratio of homologous transformants versus non-homologous transformants, preferably a 10-fold enrichment, more preferably a 1000-fold enrichment, most preferably a 10,000-fold enrichment, i.e., the ratio of transformed target cells to transformed cells. In some instances, the frequency of homologous recombination versus random integration is of the order of 1 in 1000 and in some cases as low as 1 in 10,000 transformed cells. The substantial enrichment obtained by the AMFP gene targeting vectors and methods of the invention often result in cell populations wherein about 1%, and more preferably about 20%, and most preferably about 95% of the resultant cell population contains transformed target cells wherein the AMFP gene targeting vector has been homologously integrated. Such substantially enriched transformed target cell populations may thereafter be used for subsequent genetic manipulation, for cell culture experiments or for the production of transgenic organisms such as transgenic animals or plants.

The following Examples are presented by way of example and is not to be construed as a limitation on the scope of the invention.

### EXAMPLES

### Example 1

### Inactivation of the ptch2 Locus Through the Utilization of AMFP Vectors and Methods in ES Cells

1. ptch2 Targeting Vector Construction

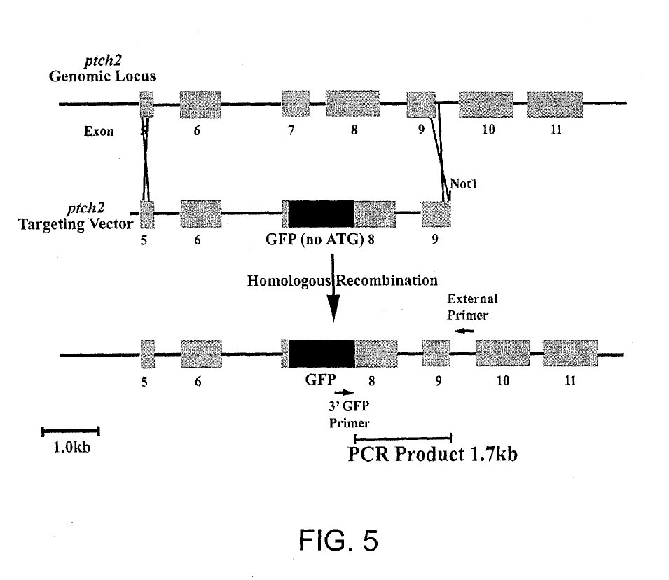

ptch2 is a transmembrane domain receptor speculated to play a role in the modulation of hedgehog signaling during embryonic development and postnatally (Motoyama, J. et al. (1998), Nat. Genet., 18, 104; Carpenter, D. et al., PNAS, 95, 13630). The ptch2 targeting vector may be constructed from a lambda phage mouse genomic DNA library utilizing a phage clone containing genomic sequences spanning exons 5 through 11, which contain transmembrane domains 2 through 8 of the ptch2 receptor (FIG. 5). Briefly, a 1.7 kb 3′ region of homology may be amplified from genomic DNA isolated from the ptch2 phage clone by PCR and flanked with Kpn1 and Not1 sites. The fragment may subsequently be subcloned into the pPolylinker plasmid and the plasmid therein after referred to as pPolylinker 1.7. A 5′ region of homology containing exons 5, 6 and the most 5′ region of exon 7 may be removed from the genomic clone with the restriction enzymes BamH1 and Nco1, filled in with Klenow fragment DNA polymerase and blunt subcloned into an Hpa1 site of pPolylinker 1.7. A DNA fragment encoding the green fluorescent protein (GFP) lacking sequences coding for an initiating methionine may be inserted between the 5′ and 3′ regions of homology to replace coding regions for transmembrane domains 2, 3 and 4, thus inactivating the receptor (FIG. 5).

2. Transformation of ES Cells with ptch2 Targeting Vector

A Not1 site present at the 3′ end of the targeting vector just downstream of the 3′ region of homology may be utilized for linearization prior to embryonic stem cell transformation. 100 ug of the AMFP gene targeting vector may be linearized, phenol/chloroform extracted, ethanol precipitated and resuspended in sterile filtered water at a concentration of 1 ug/ul prior to embryonic stem cell transformation. Stem cells are propagated at 37 deg. C., 5% CO₂ on gelatinized 10 cm plates to approximately 50% confluency in M15 media containing 15% FCS, 0.1 mM non-essential amino acids, 1 mM sodium pyruvate, 10⁻⁴M B-mercaptoethanol, 2 mM L-glutamine, 50 ug/ml penicillin, 50 ug/ml streptomycin, 1000U/ml LIF in Dulbecco's minimal essential medium (DMEM). Cells are subsequently rinsed in media-free DMEM and 8 ug linearized vector per 10 cm plate of ES cells introduced via lipofection techniques with Lipofectamine Reagent according to the manufacturer's specifications (Invitrogen, Inc.). 24-48 hours post transfection cells may be harvested for separation in a FACStar Plus cell sorter. Cell harvesting includes two rinses in sterile filtered phosphate buffered saline (PBS) followed by trypsinization in 1 ml of 0.05% trypsin/EDTA per 10 cm plate for 15 minutes. Excess trypsin is removed and cells resuspended in cell sorting buffer containing 1 mM EDTA, 25 mM HEPES, pH 7.0 and 1% dialyzed FCS in PBS at a density of 10*10⁶ cells/ml. Cells were kept on ice in 5% CO₂ prior to sorting.

3. Separation of ptch2 Targeted and Nontargeted ES Cells

ES cells transfected with the AMFP gene targeting vector may be selected for 10-12 days, harvested as described above and bulk sorted in a FACStar Plus cell sorter to separate cells expressing GFP from those which do not express it (FIG. 4). Sorted cell populations including GFP are replated at a density of 10*10⁶ cells/10 cm plate and propagated to 80% confluency for subsequent isolation of DNA and genotyping (FIG. 4).

4. Genotyping Confirmation of ptch2 Mutation by Site-specific Homologous Recombination

Genomic DNA is isolated from sorted ES cell populations by the following protocol. Cells are grown in 10 cm plates to approximately 80% confluence and 1 ml lysis buffer containing 100 mM sodium chloride, 50 mM Tris-HCl, pH 7.5, 10 mM EDTA and 0.5% sodium dodecyl sulfate (SDS) added directly to the plates. Cells are incubated for 15 minutes at room temperature, transferred to 1.5 ml Eppendorph tubes and incubated at 55 deg. C. overnight with gentle shaking. Lysates are extracted two times with an equal volume of 1:1 phenol/chloroform and one time with chloroform. Genomic DNA is precipitated with an equal volume of isopropanol. After centrifugation at 15000×G genomic DNA pellets are resuspended in 300 ul sterile filtered water.

Genomic DNA from each sample may be genotyped by PCR utilizing an oligonucleotide primer specific for sequences in the coding region of GFP and an oligonucleotide specific for sequences just downstream of the 3′ region of homology (FIG. 5). 20 pmoles of each oligonucleotide are mixed with 100 ng genomic DNA in the presence of 200 uM final concentration of each dNTP, 2.5 mM MgCl₂, 1X PCR buffer and 1U Taq DNA polymerase (Invitrogen, Inc.). Amplification is performed through application of the following cycling parameters: 94.0 deg. C. for 2 minutes followed by 35 cycles of 96 deg. C. for 30 seconds, 58 deg. C. for 30 seconds and 72 deg. C. for 2.5 minutes. Reactions are electrophoresed in parallel with 1kb ladder molecular weight standards on a 0.8% agarose gel and the gel stained with ethidium bromide for UV detection of PCR products. A 1.7 kb PCR product will be detected utilizing DNA from sample populations sorted to include GFP upon successful site-directed homologous recombination.

### Example 2

### Inactivation of the paraxis Locus Through the Utilization of AMFP Vectors and Methods in ES Cells

1. paraxis Targeting Vector Construction

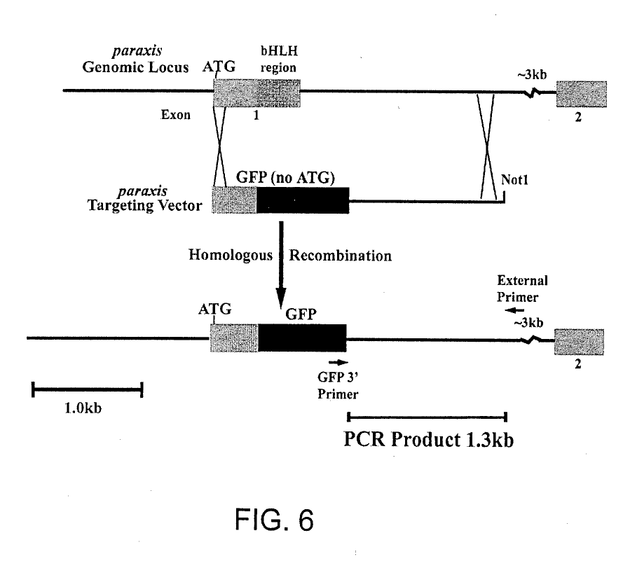

paraxis is a basic helix-loop-helix transcription factor implicated in the control of somite formation during mammalian embryogenesis (Burgess, R. et al., (1995), 168, 296; Burgess, R. et al., (1996), Nature, 384, 570; Barnes, G. L. et al. (1997), Dev. Biol., 189, 95). The construction of the paraxis targeting vector has been previously described (Burgess, R. et al., (1996), Nature, 384, 570). The paraxis genomic organization consists of two exons separated by a 5 kb intron. The first exon contains the initiating methionine codon and the basic helix-loop-helix (bHLH) domain responsible for DNA binding and dimerization. Green fluorescent protein (GFP) lacking sequences coding for an initiating methionine may be utilized to replace the majority of exon 1 as well as 5′ regions of intron 1 (FIG. 6).

2. Transformation of ES Cells with a paraxis Targeting Vector

Embryonic stem cells grown to approximately 50% confluency are transfected with 20 ug of linearized AMFP targeting vector. Transfections may be accomplished via lipofection protocols according to manufacturer's specifications (Invitrogen, Inc.). 10-12 days post-transfection cells are harvested and bulk sorted in a FACStar Plus cell sorter to separate cells not expressing GFP from those which express it. Sorted cell populations expressing GFP are replated at a density of 10*10⁶ cells/10 cm plate and propagated to 80% confluency for isolation and DNA and genotyping (FIG. 4).

3. Genotyping Confirmation of paraxis Mutation by Site-specific Homologous Recombination

Genomic DNA may be isolated from either sorted ES cell populations or unsorted negative control cells. Genomic DNA from each sample is genotyped by PCR utilizing an oligonucleotide primer specific for sequences in the 3′ region of GFP and an oligonucleotide specific for sequences just downstream and outside of the 3′ region of homology (FIG. 6). Reaction volumes and conditions are as described above with the exception of the primer annealing temperature which was 55 deg. C. Reactions are electrophoresed in parallel with 1 kb ladder molecular weight standards on a 0.8% agarose gel and the gel stained with ethidium bromide for UV detection of PCR products. A 1.5 kb PCR product detected utilizing DNA from sample populations sorted to include GFP expression indicates site-specific homologous recombination and successful gene targeting.

Having described the preferred embodiments of the present invention, it will appear to those ordinarily skilled in the art that various modifications may be made to the disclosed embodiments, and that such modifications are intended to be within the scope of the present invention. Although the invention has been described with reference to the above examples, it will be understood that modifications and variations are encompassed within the spirit and scope of the invention. Accordingly, the invention is limited only by the following claims.

What is claimed is:

1. A method for identifying a transformed cell which has undergone site-specific homologous recombination utilizing a AMFP vector comprising:
a) transforming cells with a AMFP vector designed to undergo site-specific homologous/recombination wherein the vector comprises:
a first DNA sequence which is substantially homologous to an endogenous genomic sequence present within the host genome;
a second DNA sequence which encodes a fluorescent protein selection characteristic lacking regulatory elements and sequences coding for an initiating methionine in said cells and is non-homologous to cellular endogenous genomic sequences and therefore incapable of undergoing site-specific homologous recombination;
a third DNA sequence which is substantially homologous to an endogenous genomic sequence present within the host genome and is different from the first DNA sequence; and
b) propagating cells to select for or enrich for those which have been transformed with said AMFP vector by selecting for the presence of the functional fluorescent protein selectable marker gene product of said second DNA sequence; and
c) separating cells which have said second DNA sequence encoding a functional fluorescent protein selectable marker from cells which do not have said second DNA sequence.

2. The method of claim 1, further comprising d) characterizing the genomic DNA of said cells carrying the second DNA sequence encoding a functional fluorescent protein selectable marker for the site-specific homologous recombination events which allow for modification of the cellular target DNA.

3. The method of claim 1 wherein said AMFP vector includes selectable markers which may be detected by fluorescence light emission.

4. The method of claim 1 wherein said fluorescent protein selectable markers allow for the separation of cells containing DNA encoding one marker from cells containing DNA encoding another or both markers.

5. The method of claim 1 wherein said cells are capable of homologous recombination.

6. The method of claim 1 wherein said cells are from a multicellular organism.

7. The method of claim 1 wherein said cells are from plants.

8. The method of claim 1 wherein said cells have undergone multiple rounds of site-specific homologous recombination for the purposes of multiple modifications of the endogenous cellular genome.

9. The method of claim 1 wherein said cells may be utilized to create a multicellular organism.

10. The method of claim 1 wherein said cells are embryonic stem cells.

11. An isolated AMFP gene targeting vector for site-specific homologous recombination in cells capable of undergoing homologous recombination, the vector comprising:
a first DNA sequence which is substantially homologous to cellular endogenous genomic sequences and is capable of undergoing homologous recombination in said cells,
a second DNA sequence which is nonhomologous to cellular endogenous genomic sequences, is not capable of undergoing homologous recombination in said cells, does not contain regulatory elements, encodes a fluorescent protein selectable marker lacking sequences coding for an initiating methionine and capable of allowing for the identification of cells containing said positive selectable marker,
a third DNA sequence which is substantially homologous to cellular endogenous genomic sequences and is capable of undergoing homologous recombination in said cells,
wherein the organization of said AMFP gene targeting vector in 5′ to 3′ orientation comprises: the first DNA sequence which is substantially homologous to cellular endogenous genomic DNA sequences, the second DNA sequence which does not contain regulatory elements and encodes a fluorescent protein selectable marker which lacks sequences coding for an initiating methionine, and the third DNA sequence which is substantially homologous to cellular endogenous genomic DNA sequences;
wherein the vector is capable of undergoing site-specific homologous recombination resulting in modification of cellular endogenous target genomic DNA sequences.

12. The AMFP gene targeting vector of claim 11 wherein said cellular endogenous genomic target DNA is comprised of exons and introns.

13. The AMFP gene targeting vector of claim 11 wherein said vector contains all or portions of exons and introns which are substantially homologous to cellular target genomic DNA sequences.

14. The AMFP gene targeting vector of claim 11 wherein said vector contains portions of regulatory elements which are substantially homologous to cellular target genomic DNA sequences.

15. The AMFP gene targeting vector of claim 11 wherein said vector contains alterations in sequences which are substantially homologous to cellular target genomic DNA sequences including deletions, substitutions, additions or point mutations.

16. The AMFP gene targeting vector of claim 11 wherein said fluorescent protein selectable marker encoded by said second DNA sequence is selected from DNA sequences encoding fluorescent proteins including GFP, CFP, YFP, RFP, dsRED or HcRED.

17. The AMFP gene targeting vector of claim 11 wherein said vector has lengths of homology for said first and third DNA sequences which are between about 50 bp and 50,000 base pairs.

18. The AMFP gene targeting vector of claim 11 wherein said vector results in the modification of cellular endogenous genomic target DNA sequences.

19. The AMFP gene targeting vector of claim 11 wherein said vector introduces exogenous regulatory elements into the cellular endogenous genomic target DNA sequences.

20. An enriched population of cells generated through a method according to claim 1 wherein said cells have undergone site-specific homologous recombination.

21. A non-human transgenic animal generated by the method of claim 1 wherein said animal has been generated from cells which have undergone site-specific homologous recombination.

22. A transgenic plant generated by the method of claim 1 wherein said plant has been generated from cells which have undergone site-specific homologous recombination.
